(19) United States
(12) Patent Application Publication
(10) Pub. No.: US 2024/0310361 A1
(43) Pub. Date: Sep. 19, 2024

(54) METHOD OF EVALUATING IMMUNE RESPONSE OF CELL GROUP TO TEST SUBSTANCE

(71) Applicant: CANON KABUSHIKI KAISHA, Tokyo (JP)

(72) Inventors: KOUICHI KATO, Kanagawa (JP); TAKANORI YODA, Kyoto (JP); TSUTOMU HONMA, Tokyo (JP)

(21) Appl. No.: 18/605,551

(22) Filed: Mar. 14, 2024

(30) Foreign Application Priority Data
Mar. 16, 2023 (JP) 2023-042233

(51) Int. Cl. G01N 33/50; G01N 21/64

(52) U.S. Cl. CPC G01N 33/5047; G01N 21/6428; G01N 2021/6439

(57) ABSTRACT

Provided is a method of evaluating the immune response of a cell group to a test substance, the method comprising the steps of: immobilizing a cell membrane modifier comprising a polymer having a hydrophobic chain and a hydrophilic chain on a substrate; introducing a calcium fluorescent indicator into the cell group; immobilizing the cell group on the substrate through the cell membrane modifier, adding the test substance to the cell group; and detecting the fluorescence of the cell group derived from the calcium fluorescent indicator with a detector.

### BACKGROUND

### Field of the Disclosure

The present disclosure relates to a method and a test kit for evaluating an immune response of a cell group to a test substance.

### Description of the Related Art

Animals including humans are equipped with a mechanism of defense through inactivation of a foreign substance, such as a pathogen or a toxin, which enters the body, or a cancer cell, that is, an immune system. The immune system includes an early response, which is a quick reaction to a non-specifically recognized foreign substance, that is, innate immunity, and an “antigen-specific immune response” (also called acquired immunity or adaptive immunity), which is a reaction to a foreign substance or a part thereof that is specifically recognized as an “antigen.”

An antigen-specific immune response involves T cells, B cells, and other immune cells, and is equipped with a mechanism of storing information of an antigen encountered in the past and responding quickly upon re-encounter with the same antigen. Accordingly, evaluation of an antigen-specific immune response of an immune cell is useful in examinations and studies on such diseases as infectious diseases, cancers, allergies, and autoimmune diseases, evaluation of a medicinal agent that acts on the immune system as an antigen, such as a vaccine, and the like.

As a typical method of evaluating an antigen-specific immune response of a cell, there has been known a method including stimulating a T cell or another immune cell with an antigen and detecting an immune effector molecule (for example, a cytokine such as INF-γ) secreted as a result of a response of the immune cell to the antigen.

For example, enzyme-linked immunospot (ELISpot) assay is a method in which an antibody bound to a surface of a solid phase that is an antibody against an immune effector molecule is used to capture immune effector molecules secreted from cells cultured on the solid phase and, after the cells are removed and the surface of the solid phase is washed, the molecules are visualized with use of a labeled antibody, to thereby count the cells that have secreted the molecules. In addition, FluoroSpot assay is a method of evaluating an antigen-specific immune response with the same mechanism as the mechanism of ELISpot, except that a fluorescently labeled antibody is used to visualize the immune effector molecules, and enables simultaneous detection of a plurality of types of immune effector molecules by using a plurality of labeled antibodies different from one another in fluorescence wavelength. ELISpot assay and FluoroSpot assay are widely used in medical tests and drug development because of high sensitivity and capability to evaluate on a cell-by-cell basis. For example, a test to see if there is tuberculosis infection and a check of effects of materials that are candidates for a vaccine use ELISpot assay or FluoroSpot assay.

Meanwhile, ELISpot assay and FluoroSpot assay are cumbersome in terms of operation and require proficiency at work. Further, ELISpot assay and FluoroSpot assay require cultivation of cells until immune effector molecules are secreted in an amount sufficient for visualization and, as a result, usually take 1 to 2 days or longer for a testing time. A simpler and quicker evaluation method to replace ELISpot assay and FluoroSpot assay has accordingly been sought in, for example, a case of an infectious disease or the like in which testing is required to be quick and a case of drug development in which a large number of candidate substances or specimens are required to be evaluated.

It has been known that a T cell receptor (TCR) stimulated with an antigen-MHC complex immediately increases a calcium concentration in a T cell (Non Patent Literature 1; Gillian Grafton, Leanne Thwaite, Calcium channels in lymphocytes. Immunology. 2001 October; 104(2): 119-126.). When a test substance is added to peripheral blood mononuclear cells (PBMCs) including the T cells of a subject, and such a change that an intracellular calcium concentration increases is detected, the number of the T cells each having responsiveness to the test substance can be measured in a short time period within several minutes after the stimulation with the test substance. Further, to detect a change in calcium concentration for each cell for improving measurement accuracy, the cells only need to be observed before and after antigen stimulation, or with time while the cells are immobilized so that their positions do not move during the observation. The method of measuring the immobilized cells is also reported in Non Patent Literature 2 (Nadia Anikeeva et. Al., Evaluating frequency and quality of pathogen-specific T cells. Nat. Commun. 2016 Oct. 27; 7: 13264.). According to Non Patent Literature 2, poly-L-lysine (PLL) is immobilized on an observation substrate, an anti-leucocyte function associated antigen-1 (LFA-1) monoclonal antibody is immobilized via the PLL, and T cells are immobilized on the substrate via LFA-1 molecules present on the surfaces of the T cells by the immobilized antibody. However, the LFA-1 molecule is a molecule involved in an immune synapse formed by the binding of the TCR and the antigen-MHC complex, and hence concern is raised in that the binding of the antibody to the LFA-1 molecule on the T cell affects the functions of the T cell.

As described above, a change in calcium concentration in a cell is a promising indicator that can quickly reflect the response of the cell to a test substance, but its observation has heretofore been performed while the cell is immobilized through a protein molecule on the surface of the cell. Accordingly, it has been unable to eliminate a risk in that the immobilization affects an evaluation result in a medical test or drug development.

### SUMMARY

An object of the present disclosure is to provide a method and a test kit for simply and quickly evaluating the antigen-specific immune response of a cell group. There is provided a method of evaluating an immune response of a cell group to a test substance, the method including evaluating the immune response of the cell group to the test substance based on a comparison between: first autofluorescence information which is autofluorescence information of a first sample, the first sample being prepared by adding the test substance to the cell group; and second autofluorescence information which is autofluorescence information of a second sample, the second sample serving as a control for the first sample.

Further features of the present disclosure will become apparent from the following description of embodiments with reference to the attached drawings.

### BRIEF DESCRIPTION OF THE DRAWINGS

FIG. 1A is a conceptual diagram for illustrating an example in which a cell group immobilized on a substrate having immobilized thereon a cell membrane modifier specifically reacts to a test substance to emit fluorescence derived from a calcium fluorescent indicator, the figure being a conceptual diagram of cells immobilized on the substrate.

FIG. 1B is a conceptual diagram for illustrating the example in which the cell group immobilized on the substrate having immobilized thereon the cell membrane modifier specifically reacts to the test substance to emit the fluorescence derived from the calcium fluorescent indicator, the figure being a conceptual diagram obtained by enlarging a dotted line portion in FIG. 1A.

FIG. 2 is a conceptual diagram for illustrating this embodiment.

FIG. 3 is a diagram for illustrating a system configuration of an image pickup and analysis apparatus.

FIG. 4 is a diagram for illustrating a functional configuration of a part 1A of the image pickup and analysis apparatus of FIG. 3.

FIG. 5 is a diagram for illustrating a functional configuration of a part 2A of the image pickup and analysis apparatus of FIG. 3.

FIG. 6 is a scheme chart of image analysis in which an image for cell region extraction and a fluorescence image that have been photographed by the image pickup and analysis apparatus are used, and an image processing device takes information out of the images, plots, and visualizes.

FIG. 7 is a bright-field image of PBMCs immobilized on a BAM-modified glass bottom dish.

FIG. 8 is a fluorescence image of the immobilized cells shown in FIG. 7 before the addition of a calcium ionophore.

FIG. 9 is a fluorescence image of the immobilized cells shown in FIG. 7 after the addition of the calcium ionophore.

FIG. 10 shows the result of a comparison between a change in number of cells each having a fluorescence intensity equal to or more than an arbitrarily set threshold with time in the presence of antigen stimulation and that in the absence of the stimulation.

### DESCRIPTION OF THE EMBODIMENTS

More specific configuration examples of the present disclosure are described below, but the present disclosure is not limited to the following methods.

According to one embodiment of the present disclosure, there is provided a method of evaluating an immune response of a cell group to a test substance, the method comprising: an immobilizing step of immobilizing a cell membrane modifier comprising a polymer having a hydrophobic chain and a hydrophilic chain on a substrate; an introducing step of introducing a calcium fluorescent indicator into the cell group; an immobilizing step of immobilizing the cell group on the substrate through the cell membrane modifier; an adding step of adding the test substance to the cell group; and an detecting step of detecting fluorescence of the cell group derived from the calcium fluorescent indicator with a detector.

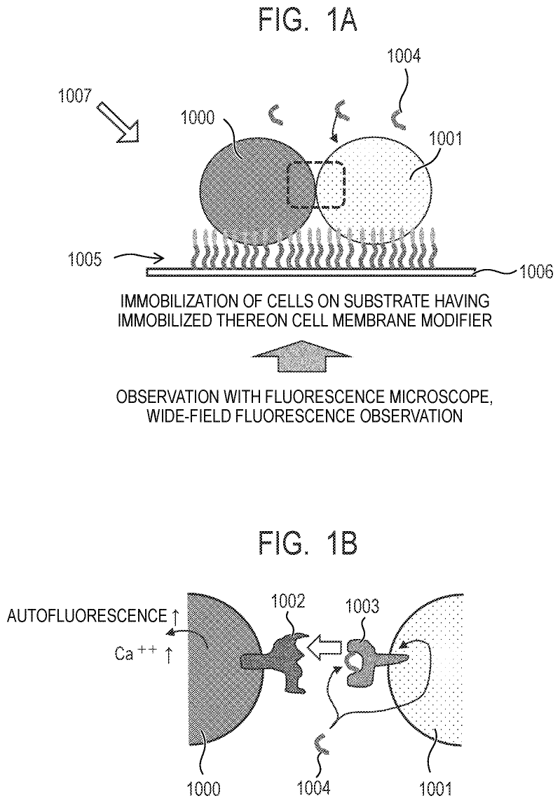

An example of a cell group showing an immune response to a test substance is illustrated in each of FIG. 1A and FIG. 1B. The cell group illustrated in each of FIG. 1A and FIG. 1B includes a T cell 1000 and an antigen presenting cell 1001. A calcium fluorescent indicator 1007 is introduced into such cell group. The respective cells are immobilized on a substrate 1006 through a cell membrane modifier 1005. A test substance 1004 is presented on a major histocompatibility complex (MHC) 1003 on the antigen presenting cell 1001. When the T cell has a T cell receptor (TCR) 1002 specific to a complex of the test substance 1004 and the major histocompatibility complex 1003, those are bound together to activate the T cell 1000. As a result, a calcium ion concentration in the T cell 1000 increases to cause the calcium fluorescent indicator 1007 to emit fluorescence. The T cell immune response of the cell group to the test substance is evaluated by detecting the fluorescence with a detector (not shown).

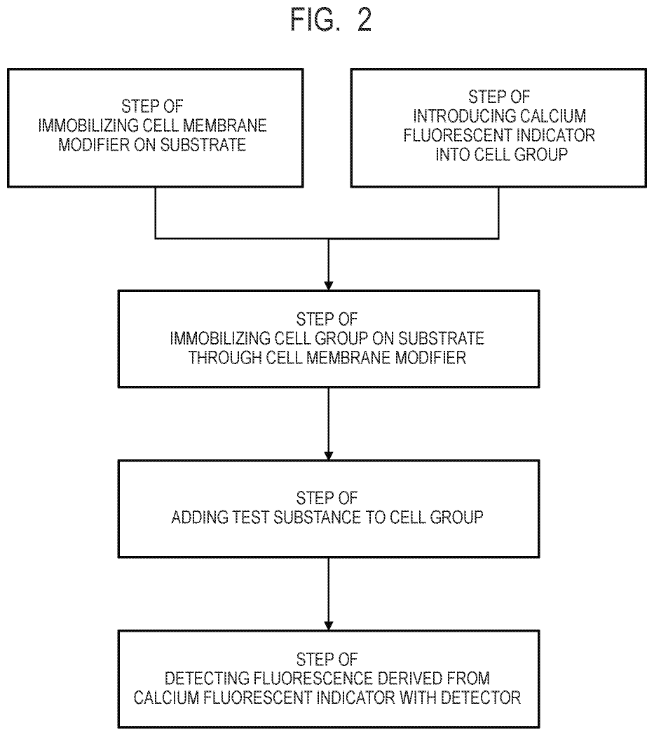

A conceptual diagram of this embodiment is illustrated in FIG. 2. That is, the method of this embodiment includes the steps of: immobilizing the cell membrane modifier on the substrate; introducing the calcium fluorescent indicator into the cell group; immobilizing the cell group on the substrate through the cell membrane modifier; adding the test substance to the cell group; and detecting the fluorescence derived from the calcium fluorescent indicator with the detector. Each of the step of immobilizing the cell membrane modifier on the substrate and the step of introducing the calcium fluorescent indicator into the cell group only needs to be performed before the immobilization of the cell group, and it does not matter which one of these steps is performed first.

### (Cell Membrane Modifier)

The cell membrane modifier includes the polymer having the hydrophobic chain and the hydrophilic chain. The hydrophobic chain serves as an anchor for a cell membrane, and the hydrophilic chain thereof improves the water solubility of the modifier. The hydrophobic chain is selected from a saturated or unsaturated hydrocarbon chain, and a lipid and a complex lipid chain for forming the cell membrane, and is preferably selected from the group consisting of saturated or unsaturated hydrocarbon chains each having 6 or more and 22 or less carbon atoms. The most preferred hydrophobic chain is an oleyl group. The hydrophilic chain is at least one kind selected from the group consisting of: a protein; an oligonucleotide; a polymer or copolymer of a derivative of each of glycolic acid, lactic acid, and P-dioxane; an oligopeptide; a polypeptide; a polyamide; a polyalkylene glycol; and a polysaccharide, and is preferably a polyethylene glycol (PEG) (polyoxyethylene structure). A PEG having a molecular weight of from 1 KDa to 10 KDa may be utilized in a PEG chain, and a PEG having a molecular weight of 4 KDa is preferably utilized. Further, the hydrophilic chain may have a functional group, and the hydrophilic chain preferably has an active ester group at a terminal thereof.

A molecule represented by the following formula (1) may be preferably used as the cell membrane modifier. The molecule is commonly referred to as “biocompatible anchor for membrane (BAM).”

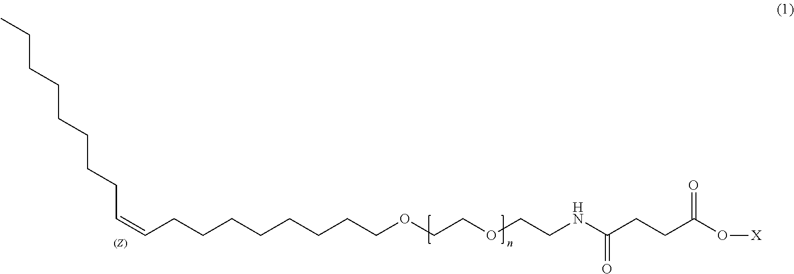

In the formula (1), X represents a reactive group, and “n” represents an integer of 20 or more and 200 or less. It is preferred that-X represent a group represented by the formula (2).

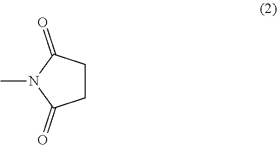

That is, specifically, for example, a molecule represented by the following formula (3) (sometimes referred to as “BAM-NHS” herein) may be used as the cell membrane modifier:

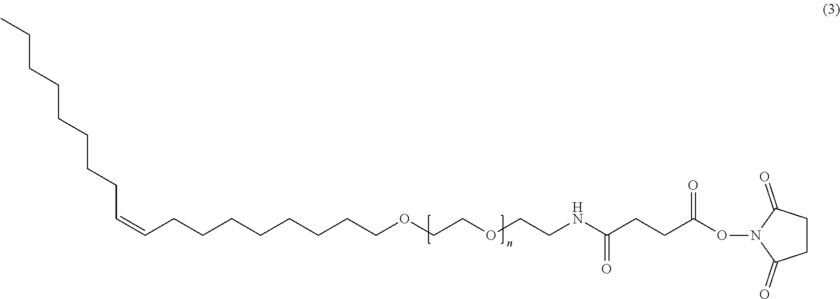

in the formula (3), “n” represents an integer of 20 or more and 200 or less.

The hydrocarbon chain of the cell membrane modifier enters the cell membrane of a cell in the cell group to serve as an anchor. Accordingly, the modifier can be immobilized on the substrate without any damage to the cell. In this embodiment, the cells of the cell group preferably include a T cell, and for example, PBMCs are used as the group. However, the T cell and the PBMCs are each a floating cell. Accordingly, unless the floating cell is immobilized on the substrate, the cell moves during its observation, and is hence hard to observe. However, the use of the cell membrane modifier can prevent the movement. In addition, as illustrated in each of FIG. 1A and FIG. 1B, a T cell immune response requires antigen presentation by an antigen presenting cell. To cause the immune response, the individual cells of the cell group are preferably immobilized under the state of being brought into contact with each other. The use of the cell membrane modifier can achieve the immobilization.

### (Substrate Having Bound thereto Cell Membrane Modifier)

The substrate is preferably a material having high transparency suitable for cell observation, and for example, glass or a transparent resin, such as polystyrene, polycarbonate, polyethylene terephthalate, or acrylic, may be used.

The cell membrane modifier may be bound to the surface of the substrate through a functional group present on the surface of the substrate, preferably through an amino group. To arrange a functional group on the surface of the substrate, at least one kind of a protein, a peptide, a silane coupling agent, or a polymer having a functional group may be used while binding to the surface of the substrate. An example of the protein to be bound to the substrate for arranging a functional group on the surface of the substrate is bovine serum albumin (BSA).

As an example of the production of a substrate having bound thereto a cell membrane modifier, BSA was caused to physically adsorb to the glass portion of a glass bottom dish, and BAM-NHS was able to be bound to the BSA bound onto the substrate.

Although a concentration of 1% or more is often adopted as the concentration of a BSA solution when the BSA is caused to physical adsorb to the glass portion of the glass bottom dish, the BSA concentration is arbitrary as long as the BSA can be sufficiently bound. At the time of the immobilization of PBMCs, the PBMCs are characterized in that their cell diameters are small, and hence the consideration of conditions for modification with a BAM suitable for the immobilization of the PBMCs has been required. As a result, it has been found that a cell membrane modifier-bound substrate on which the PBMCs can be immobilized is obtained by: adding (10 μL of) a solution of a BAM in PBS at a concentration in the range of from 30 μM to 900 μM to a BSA-coated glass bottom dish; and subjecting the solution to a static reaction at room temperature for 15 minutes. Further, the concentration of the solution of the BAM in PBS suitable for immobilization at a cell density suitable for a T cell evaluation is from 100 μM to 900 μM, preferably 300 μM.

### (Cell Group)

The cell group only needs to be a group in which a cell serving as a measurement object is present, and the group preferably includes a T cell.

PBMCs collected from human blood may be utilized as they are as a cell group that may be used in a clinical evaluation. Alternatively, PBMCs increased in lymphocyte ratio by preculturing PBMCs to remove adhering cells, to thereby recover floating lymphocytes may be used.

Alternatively, a cell group increased in ratio of mature T cells (CD3-positive cells), CD8-positive T cells, or CD4-positive T cells, which have been purified from human blood by an existing method, may be utilized.

Alternatively, a cell group including a cell derived from a stem cell such as an iPS cell or from a blood stem cell may be used.

When a CD4-positive T cell is evaluated, it is effective that the cell group includes an antigen presenting cell having an MHC class 2 molecule.

In addition, a cell group including a cell or a cell line derived from an animal except a human, or a T cell or a T cell line may be utilized.

### (Calcium Fluorescent Indicator)

Any indicator may be used as the calcium fluorescent indicator as long as a change in intracellular calcium concentration can be observed with a desired detector or microscope. Examples thereof include Fluo-4, Fura-2, Quin 2, Fura 8, Fluo 3, Indo 1, Rhod 2, Rhod 3, Cal-500, Cal-520, Cal-590, Cal-630, Cal-670, Cal-770, and Cal Red R525/650 AM.

### (Test Substance)

Examples of the test substance include an antigen, an antigenic protein, an antigenic peptide, a vaccine, and a pathogen. Alternatively, peptides derived from those test substances are usable.

What is capable of stimulating the T cell receptor (TCR) of a CD8-positive T cell is an MHC class 1 molecule to which a peptide derived from a test substance and formed from 8-10 amino acids is bound. What is usable as a test substance is a peptide that is derived from a test substance and that binds to an MHC class 1 molecule prepared in advance. As another form of test substance, a test substance formed from a protein or a polypeptide is usable, and this test substance may be introduced into a cell to receive processing inside the cell so that a peptide derived from the test substance is bound to an MHC molecule for use in an evaluation. As still another form of test substance, a test substance holding antigen genetic information (for example, an mRNA vaccine) is usable. This test substance may be introduced into a cell to express a protein and receive processing inside the cell so that the processed test substance is bound to an MHC molecule for use in an evaluation.

What is capable of stimulating the TCR of a CD4-positive T cell is an MHC class 2 molecule to which a peptide derived from a test substance and formed from 12-24 amino acids is bound. In the evaluation of a CD4-positive T cell as well, a peptide that is derived from a test substance and that binds to an MHC class 2 molecule prepared in advance is usable, and a test substance formed from a protein or a polypeptide and a test substance holding antigen genetic information are usable in the same manner as described above. However, an MHC class 2 molecule is expressed only on an antigen presenting cell, and coexistence with a cell expressing the molecule is accordingly required.

### (Detector)

Any detector may be utilized as the detector as long as the detector can simultaneously measure position information, time information, and fluorescence intensity information for the plurality of cells of the immobilized cell group. For example, a general-purpose microscope, such as an inverted fluorescence microscope, a confocal microscope, or a multiphoton excitation microscope for acquiring a fluorescence image, may be used.

Alternatively, an image acquisition device having a field wider than that of a typical microscope may be used. The term “wide field” as used herein means that the range of an area that can be photographed in one shot is preferably from 1 mm² to 220 mm², more preferably from 5 mm² to 220 mm², and for example, an image acquisition device using a digital camera may be used. For example, EOS R5 (product name) manufactured by Canon Inc. may be utilized as the digital camera.

In this embodiment, many cells need to be observed at a time in some cases. For example, the number of T cells in PBMCs each responding to the test substance is assumed to be from about 0.1% to about 1% of the total number of the T cells therein. If the number is regarded as 0.1%, the responses of about 100 T cells can be detected by simultaneously observing at least about 10⁵ cells. To simultaneously observe about 10⁵ cells, a wide-field image acquisition device with which a region of about 50 mm² can be observed may be suitably used.

### (Configuration of Image Pickup and Analysis Apparatus)

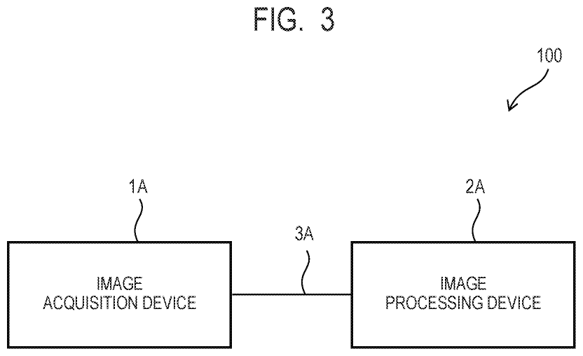

An example of the entire configuration of an image pickup and analysis apparatus 100, which is a detector that acquires fluorescence information in a wide field, is illustrated in FIG. 3. The apparatus is an apparatus that acquires an image of cells and analyzes the acquired image to analyze a change in fluorescence intensity of each cell.

As illustrated in FIG. 3, the image pickup and analysis apparatus 100 is formed by connecting an image acquisition device 1A and an image processing device 2A to each other through an interface such as a cable 3A so as to enable data transmission and reception therebetween. A connection method between the image acquisition device 1A and the image processing device 2A is not particularly limited. For example, the image acquisition device and the image processing device may be connected to each other by a local area network (LAN), or may be connected to each other in a wireless manner.

The image acquisition device 1A acquires an image of cells on a cell culture vessel that is placed on a placement stage, and transmits the image to the image processing device 2A.

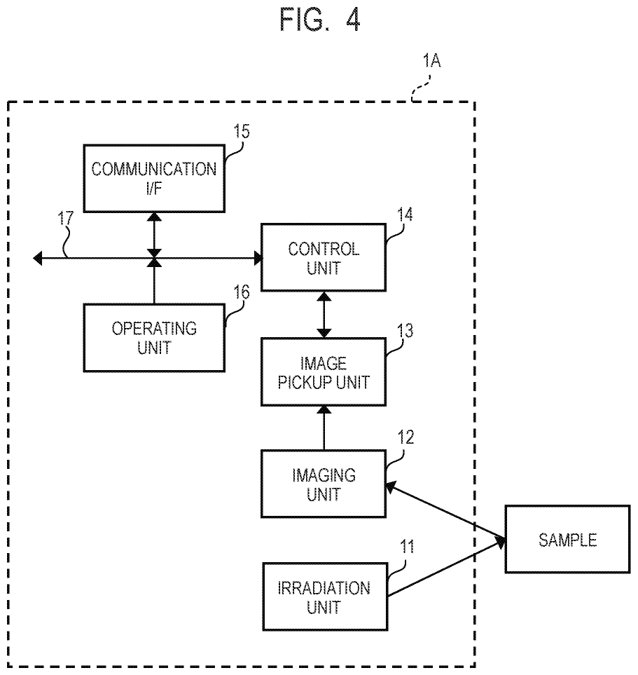

As illustrated in FIG. 4, the image acquisition device 1A includes an irradiation unit 11, an imaging unit 12, an image pickup unit 13, a control unit 14, a communication I/F 15, an operating unit 16, and the like, and the respective units are connected through a bus 17. The irradiation unit 11 is formed of a light source filter or the like for fluorescence observation and a white light source for cell morphology observation, and irradiates a cultured cell placed on the placement stage with light. The imaging unit 12 is formed of a lens, a filter, and the like, and forms an image of transmitted light, reflected light, or fluorescence that is emitted from the cells on the cell culture vessel in accordance with the applied light. The image pickup unit 13 is a camera that includes a complementary MOS (CMOS) sensor and the like, and picks up an image to be formed on an imaging surface by the imaging unit to generate digital image data (image data of R, G, and B) on the image. The control unit 14 includes a central processing unit (CPU), a random access memory (RAM), and the like, and executes various kinds of processing in cooperation with various programs installed therein. The communication I/F 15 transmits the generated image data on the image to the image processing device 2A. In this embodiment, the image acquisition device 1A includes a bright-field unit in which irradiation means and imaging means that are suitable for bright-field observation are combined, and a fluorescence unit in which irradiation means and imaging means that are suitable for fluorescence observation are combined. The bright field or the fluorescence can be switchable by switching the unit.

The image processing device 2A is an image processing device for medical use that analyzes an image transmitted from the image acquisition device 1A, to thereby calculate a feature amount quantitatively indicating an expression level of a specific biological substance in a cell to be observed on the cell culture vessel, and outputs the calculated feature amount.

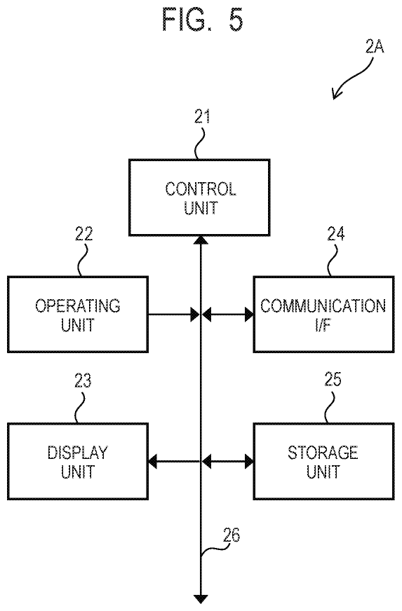

FIG. 5 is an illustration of a functional configuration of the image processing device. The image processing device 2A includes a control unit 21, an operating unit 22, a display unit 23, a communication I/F 24, a storage unit 25, and the like, and the respective units are connected through a bus 26.

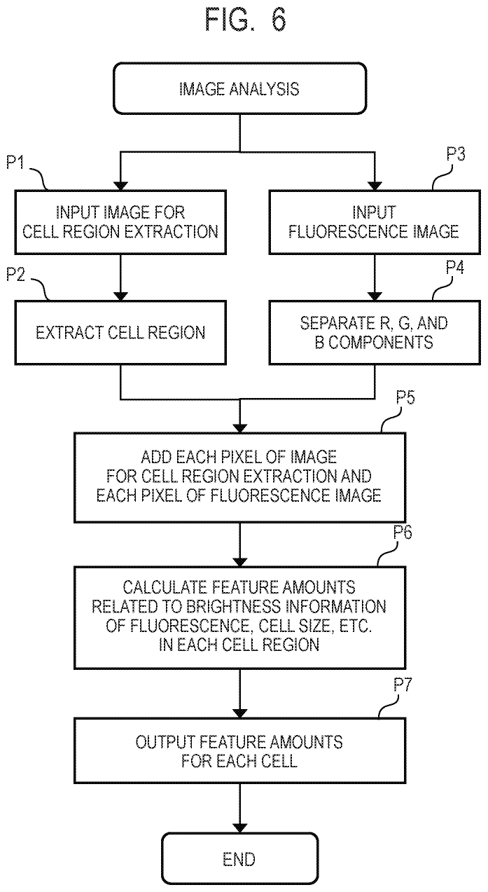

The control unit 21 includes a central processing unit (CPU), a random access memory (RAM), and the like. Various kinds of processing are executed in cooperation with various programs stored in the storage unit 25, and operations of the image processing device are centrally controlled. For example, the control unit 21 executes image analysis processing (see FIG. 6) in cooperation with a program stored in the storage unit 25, and thus implements functions as extraction means, fluorescent bright spot extraction means, region estimation means, feature amount calculation means, and determination means.

The operating unit 22 includes a keyboard provided with character input keys, number input keys, various function keys, and the like and a pointing device such as a mouse, and outputs a pressing signal of a key pressed on the keyboard and an operation signal generated by the mouse to the control unit 21 as input signals.

The display unit 23 includes, for example, a monitor such as a cathode ray tube (CRT) or a liquid crystal display (LCD), and displays various screens in accordance with an instruction of a display signal input from the control unit 21. In this embodiment, the display unit 23 functions as output means for outputting the calculated feature amount.

The communication I/F 24 is an interface for transmitting and receiving data to and from an external device such as the image acquisition device 1A. The communication I/F 24 functions as input means for a bright-field image and a fluorescence image. In this embodiment, the communication I/F 24 functions as input means.

The storage unit 25 is formed of, for example, a hard disk drive (HDD) or a semiconductor-based nonvolatile memory. The storage unit 25 stores various programs, various kinds of data, and the like as described above. For example, the storage unit 25 stores various kinds of data such as a magnification table to be used in image analysis processing described later.

In addition, the image processing device 2A may include a LAN adapter, a router, and the like and may be configured to be connected to an external device through a communication network such as a LAN.

The image processing device 2A in this embodiment performs analysis through use of the image for cell region extraction and the fluorescence image that have been transmitted from the image acquisition device 1A.

The fluorescence image is an image obtained by irradiating cells having certain fluorescence activity with excitation light having a predetermined wavelength in the image acquisition device 1A to cause a fluorescent substance derived from the calcium fluorescent indicator in the cell to emit light, and subjecting the emitted light which is fluorescence to magnification, imaging, and photographing through a cut filter for a wavelength equal to or more than a light source wavelength.

The image for cell region extraction is an image from which each cell region can be extracted by image processing. Examples thereof include a bright-field image acquired through magnification, imaging, and photographing in a bright field in the image acquisition device 1A, and a fluorescence photography image acquired by applying excitation light of a predetermined wavelength to cause a fluorescent substance in the cell to emit light, and magnifying, imaging, and photographing the emitted light which is fluorescence.

A flow chart for performing image analysis processing with use of the acquired images is illustrated in FIG. 6. The following processing steps in FIG. 6 are all executed in the image processing device 2A.

First, in Step P1, an image for cell region extraction sent from the image acquisition device 1A is input by the communication I/F 24. Subsequently, in Step P2, a cell region is extracted from the image for cell region extraction, and labeling processing is executed to label each cell.

Meanwhile, in Step P3, a fluorescence image sent from the image acquisition device 1A is input by the communication I/F 24. In Step P4, the fluorescence image may be analyzed by being separated into R, G, and B components as required.

In Step P5, a calculation of adding each pixel of the image for cell region extraction and each pixel of the fluorescence image is performed and, from the resultant addition image, information on a fluorescence color in each cell region and a feature amount related to the cell region are calculated in the subsequent step of Step P6. In Step P7, the feature amount calculated in the processing step described above is output for each cell. The output result can be displayed on the display unit 23 of the image processing device 2A.

### (Acquisition of Bright-field Image)

A method of acquiring a bright-field image for identifying the position of a cell is, for example, as follows: light from visible light sources having various wavelengths or a white light source obtained by mixing the sources is applied to the cell in parallel to the optical axis of a lens, or at an angle that is not parallel to but still is not perpendicular to the optical axis, and light reflected from the cell or light diffracted therefrom by birefringence, and interference between the reflected light and the diffracted light are detected.

### (Fluorescence Image Acquisition)

As a method of acquiring a fluorescence image, a light source having a center wavelength that is a wavelength of a unicolor from ultraviolet light to visible light as a fluorescence excitation light source wavelength is applied to a sample in parallel to the optical axis of a lens, or at an angle that is not parallel to but still is not perpendicular to the optical axis, to thereby excite fluorescence of the sample. The fluorescence generated by the excitation is detected through a cut filter installed in front of or behind the lens or the like on the observation side of the lens or the like. As cut filters for excitation light and for fluorescence observation, cut filters for the two wavelengths are selected so that a part of the excitation light source wavelength does not pass through a fluorescence cut filter on an observation side.

### (Analysis and Visualization)

An analysis and visualization method is a method including visualizing pieces of information on each cell, such as the brightness information of each color and cell size information, as a one-dimensional, two-dimensional, or three-dimensional graph in which the pieces of information are each indicated by one axis.

An example thereof is a method including visualizing such information through use of a histogram in the case of a one-dimensional graph, or a scatter plot in the case of a two-dimensional or three-dimensional graph. The information on each cell may be visualized through use of, for example, a probability density function plot such as kernel density estimation or a heatmap instead of the histogram or the scatter plot.

### (Brightness Information)

The brightness information is color information acquired by describing, in a certain color space, information of acquired fluorescence, and is coordinates in the color space. The brightness information may be described with use of information on one component out of R, G, and B in an RGB color space, which is an example of the color space, or with use of a Lab color space or an HSV color space. However, a color space other than those may also be used to describe the brightness information.

### (Cell Shape Information)

Cell shape information is information on a parameter that can describe the shape of a cell. An example thereof is information on a cell size, a cell shape, or a cell thickness, but any information other than such information may be used as long as the information can be acquired from an image.

### (Evaluation Method)

An evaluation is performed based on the number of the cells each of which has emitted the fluorescence derived from the calcium fluorescent indicator through the addition of the test substance. When the number is equal to or more than a certain number or ratio, it can be evaluated that the cell group has an immune response to the test substance.

A method of measuring the number of the cells each having reactivity to the test substance includes: identifying the cells each having a fluorescence intensity equal to or more than a cutoff arbitrarily set in an image pickup field; and measuring their number. In addition, the image processing and the measurement to be performed herein may be performed with general-purpose image analysis software.

As an example of the analysis, the following method is used: fluorescence image information before antigen stimulation is subtracted from fluorescence image information an arbitrary time period after the antigen stimulation to provide a fluorescence image as a difference; and an arbitrary threshold is set in the image, followed by the measurement of the cell number.

### (Test Kit)

The present disclosure provides, as a further embodiment, a test kit for evaluating the immune response of a cell group to a test substance, the kit including: a substrate having immobilized thereon a cell membrane modifier including a polymer having a hydrophobic chain and a hydrophilic chain; and a calcium fluorescent indicator. A molecule represented by the following formula (1) may be used as the cell membrane modifier. In addition, the kit may include, as the test substance, any one selected from an antigen, an antigenic protein, an antigenic peptide, a vaccine, and a pathogen. A material to be incorporated into the kit is not particularly limited, and the kit may include a reagent, a container, or the like required at the time of the introduction of the calcium fluorescent indicator into the cell group, at the time of the immobilization of the cell group on the substrate, or at the time of the detection of the fluorescence derived from the calcium fluorescent indicator. Further, the kit may include a casing for including such reagent, container, or the like, an instruction manual, or the like.

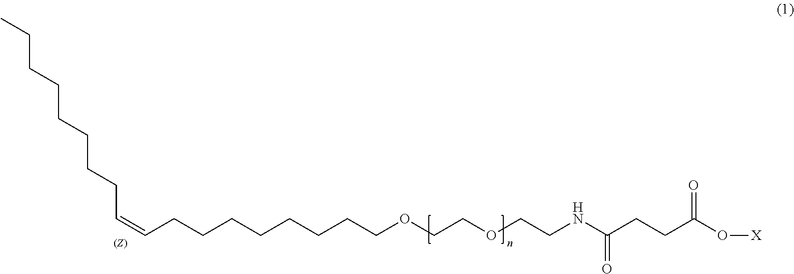

In the formula (1), X represents a reactive group, and “n” represents an integer of 20 or more and 200 or less.

### [EXAMPLES]

Examples of the present disclosure are described.

In the following Examples, specific examples of evaluating an immune response to a test substance by using PBMCs as a cell group are given. However, reagents and reaction conditions described in the following Examples may be changed and those changes are to be encompassed in the scope of the present disclosure. Accordingly, the following Examples are given for the purpose of helping understanding of the present disclosure, and are not in any way to limit the scope of the present disclosure.

### (Example 1)

### Production of BAM-modified Glass Bottom Dish

200 Microliters of a 1% solution of BSA in PBS subjected to filtration sterilization was dropped into the glass portion of a 3.5-centimeter glass bottom dish (its glass bottom was a circular shape having a diameter of 1.6 mm), and the dish was left at rest in an incubator at 37° C. overnight. The glass portion was washed with 200 μL of sterile water three times, and then the glass bottom dish was air-dried in a clean bench. 6 Milligrams of a BAM (manufactured by Yuka Sangyo Co., Ltd., product number: SUNBRIGHT OE-040CS) was added to 50 μL of DMSO to prepare a 30 mM solution of the BAM in DMSO. 10 Microliters of a solution (final concentration: 300 μM) obtained by diluting the 30 mM solution of the BAM in DMSO to 1/100 with PBS was added to the BSA-coated glass bottom dish, and the dish was left at rest at room temperature for 15 minutes. The resultant was washed with 200 μL of sterile water three times, and then the surface of the glass bottom dish was air-dried. The BAM-modified glass bottom dish was stored airtight at 4° C. until its use while a desiccant was sealed.

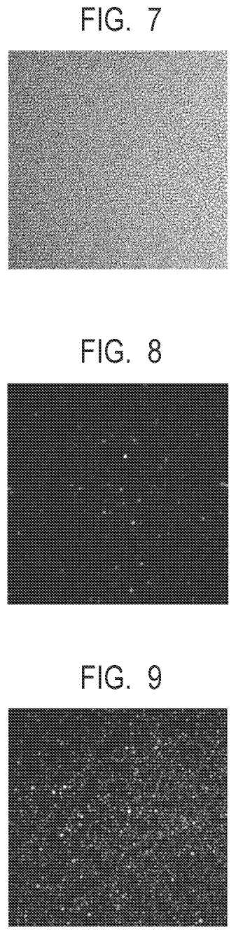

The manner in which PBMCs are immobilized on the BAM-modified glass bottom dish is illustrated in FIG. 7. A method for the immobilization is as described in Example 2.

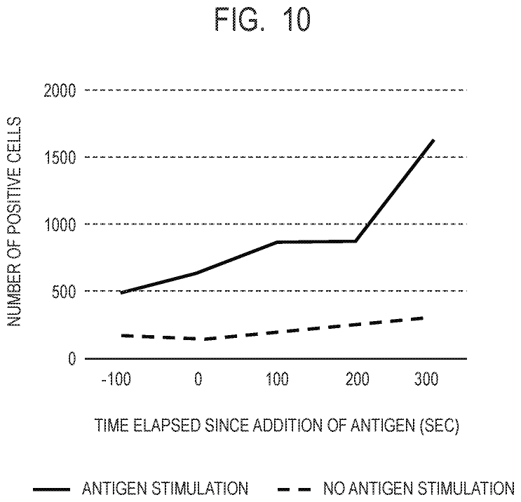

It is found from FIG. 7 that the PBMCs can be immobilized under a dense state at positions subjected to the BAM modification. To observe the reactivity of a T cell to a test substance, an opportunity in which a cell that presents an antigen and the T cell that recognizes the antigen are brought into contact with each other is needed, and hence the PBMCs need to be immobilized in such a dense manner that the individual cells are brought into contact with each other. It was able to be recognized that the cells were immobilized so as to satisfy the condition. In addition, to recognize whether or not fluorescence derived from calcium was able to be observed with the immobilized cells, a calcium ionophore (A23187) was added at a final concentration of 1 μg/mL. As a result, fluorescence was able to be observed in each of substantially all the immobilized PBMCs. FIG. 8 is a fluorescence image before the addition of the calcium ionophore, and FIG. 9 is a fluorescence image after the addition.

In this Example, the fluorescence images were acquired with an inverted microscope (TE200-U, manufactured by Nikon Corporation), a CCD camera (EM-CCD DIGITAL CAMERA/C9100-13, manufactured by Hamamatsu Photonics K.K.), and a filter set (excitation: from 450 nm to 490 nm, DM: 505 nm, fluorescence: 520 nm).

### (Example 2)

### Preparation of Fluo-4-stained PBMC cells

### (1) Thawing and Preculturing of Cells

Cells that were used were peripheral blood mononuclear cells (PBMCs) (manufactured by CTL). The PBMCs cryopreserved in a liquid nitrogen tank were quickly defrosted in a water bath at 37° C., and then 10 mL of a 1×CTL Anti-Aggregate Wash solution at 37° C. was added thereto while the cells were stirred. The mixture was centrifuged at 300 G for 8 minutes. The supernatant was removed, and 10 mL of the 1×CTL Anti-Aggregate Wash (trademark) solution at 37° C. was added to the residue again, followed by centrifugation at 300 G for 8 minutes. After that, the supernatant was removed. The cell pellet was suspended in 3 mL of CTL Test medium and an entire amount was seeded in one well of a six-well plate and cultured overnight in a 5% CO₂ incubator at 37° C.

CTL Anti-Aggregate Wash, 20x (manufactured by CTL) diluted to 1/20 with RPMI1640 medium was used as the 1×CTL Anti-Aggregate Wash solution. A test medium obtained by adding a 1/100 amount of a 200 mM L-glutamine solution was used as the CTL Test medium.

### (2) Fluo-4 Staining of Cells

Floating PBMCs were recovered from the PBMCs cultured overnight, and 7 mL of PBS was added thereto, followed by centrifugation at 300 G for 8 minutes. The supernatant was removed, and the residue was suspended in 10 mL of PBS, followed by the counting of a cell number. The cell suspension was centrifuged at 300 G for 8 minutes again, and then the supernatant was removed, followed by the staining of the PBMCs with Fluo-4 through use of Calcium Kit-Fluo 4 (manufactured by Dojindo Laboratories, product number: CS22) in accordance with the manufacturer's manual.

After the staining, the cells were suspended in a probenecid-added recording medium at 37° C. (mixed liquid of 5 ml of a recording medium (2X), 5 mL of pure water, and 50 μL of 250 mmol/l probenecid, reagent for forming Calcium Kit-Fluo 4) so that a cell concentration became 1×10⁷ cells/mL (referred to as “Fluo-4-stained PBMCs”).

### (3) Preparation of Fluo-4-stained PBMCs immobilized on Substrate Having Immobilized thereon Cell Membrane Modifier

80 Microliters of the Fluo-4-stained PBMCs were added to the BAM-modified sites of the BAM-modified glass bottom dish, and the dish was left at rest for 15 minutes. About 200 μL of the probenecid-added recording medium was quietly added to the dish, and the dish was left at rest in a 5%CO2 incubator at 37° C. until the start of measurement. The cells can be immobilized as shown in FIG. 7.

### (Example 3)

### Calcium Imaging

The probenecid-added recording medium was removed from the immobilized PBMCs stored in the CO₂ incubator immediately before the measurement, and 100 μL of the probenecid-added recording medium warmed to 37° C. was newly added thereto.

The fluorescence observation of the immobilized cells was performed with an inverted microscope (TE200-U, manufactured by Nikon Corporation), a CCD camera (EM-CCD DIGITAL CAMERA/C9100-13, manufactured by Hamamatsu Photonics K.K.), and a filter set (excitation: from 450 nm to 490 nm, DM: 505 nm, fluorescence: 520 nm or more). One fluorescence image was acquired before the addition of a test substance. Next, 100 μL of a solution of the test substance was added to the cells, and a fluorescence image was acquired at an arbitrary time thereafter.

Herein, 2X CMV pp65 (manufactured by Cellular Technology Limited) (concentration: 10 nM) was used as the test substance. It has been recognized in advance by the manufacturer through an ELISpot assay that the test substance is a peptide showing an extremely strong reaction in the PBMCs used in this Example.

### Analysis Method and Results

Analysis was performed with fluorescence images acquired before the addition of the test substance, immediately after the addition of the test substance, 100 seconds thereafter, 200 seconds thereafter, and 300 seconds thereafter. The number of cells (number of positive cells) having an average fluorescence intensity in the threshold range of from 50 to 255 was measured in each image. The results are shown in FIG. 10.

When the test substance was added, a significant increase in number of the positive cells with time was observed. Meanwhile, when the test substance was not added, no significant increase in number of the positive cells was observed. It was able to be recognized from the foregoing results that the immobilized PBMCs showed an immune response to stimulation by the addition of the test substance to cause an increase in intracellular calcium concentration within several minutes, and hence the reactivity of each of the cells to the added test substance was able to be measured in a short time period.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

When a substrate is subjected to immobilization modification with a cell membrane modifier including a polymer having a hydrophobic chain and a hydrophilic chain at an appropriate density, a cell group can be quickly immobilized on the substrate, and hence can be observed with time without any change in cell position. In addition, the immobilization has small influences on the functions of cells. When an intracellular calcium fluorescent indicator was used in the immobilized cell group, the immune response of the cell group reacting with a test substance was able to be evaluated with a fluorescence microscope.

A measuring system that collectively photographs many cells at a time with a high-pixel wide-field image pickup device may be utilized.

As compared to such a cell immobilization method including utilizing an antibody as seen in the related art, in which cells are immobilized through a cell membrane protein that may show different expression amounts for the respective cells, an antigen-presenting cell except a T cell, a B cell, and the like can be immobilized as in the T cell irrespective of the kinds of cells because the cell membrane modifier is directly bound to a cell membrane. Accordingly, there is provided a more appropriate method for the performance of an evaluation involving an interaction between various cells.

While the present disclosure has been described with reference to embodiments, it is to be understood that the disclosure is not limited to the disclosed embodiments. The scope of the following claims is to be accorded the broadest interpretation so as to encompass all such modifications and equivalent structures and functions.

This application claims the benefit of priority from Japanese Patent Application No. 2023-042233, filed Mar. 16, 2023, which is hereby incorporated by reference herein in its entirety.

What is claimed is:

1. A method of evaluating an immune response of a cell group to a test substance, the method comprising:
a first immobilizing step of immobilizing a cell membrane modifier comprising a polymer having a hydrophobic chain and a hydrophilic chain on a substrate;
an introducing step of introducing a calcium fluorescent indicator into the cell group;
a second immobilizing step of immobilizing the cell group on the substrate through the cell membrane modifier;
an adding step of adding the test substance to the cell group; and
a detecting step of detecting fluorescence of the cell group derived from the calcium fluorescent indicator with a detector.

2. The method according to claim 1, wherein the cell membrane modifier has, as the hydrophobic chain, a saturated or unsaturated hydrocarbon chain having 6 or more and 22 or less carbon atoms.

3. The method according to claim 1, wherein the cell membrane modifier has, as the hydrophilic chain, polyethylene glycol.

4. The method according to claim 1, wherein the cell membrane modifier is represented by the formula (1): 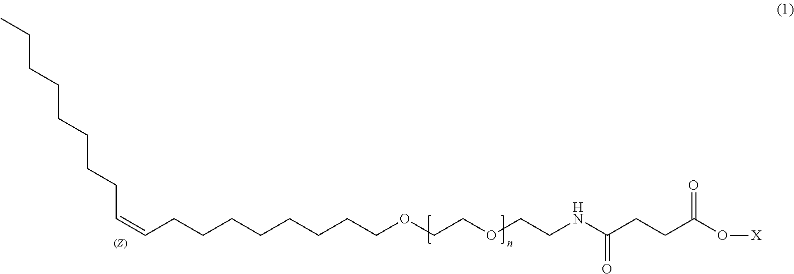
in the formula (1), X represents a reactive group, and “n” represents an integer of 20 or more and 200 or less.

5. The method according to claim 4, wherein-X in the formula (1) is a group represented by the formula (2).

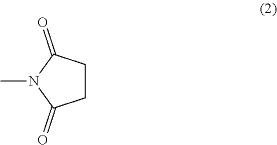

6. The method according to claim 1, wherein the cell group is peripheral blood mononuclear cells (PBMCs).

7. The method according to claim 1, wherein the cell group is floating lymphocytes in peripheral blood mononuclear cells (PBMCs).

8. The method according to claim 1, wherein the test substance is an antigen, an antigenic protein, an antigenic peptide, a vaccine, or a pathogen.

9. The method according to claim 1, wherein the test substance is an antigenic peptide capable of binding to a major histocompatibility complex (MHC) class 1 molecule.

10. The method according to claim 1, wherein the detector is a camera capable of photographing an area of from 1 mm² to 220 mm² in one shot.

11. A test kit for evaluating an immune response of a cell group to a test substance, the test kit comprising:
a substrate having immobilized thereon a cell membrane modifier comprising a polymer having a hydrophobic chain and a hydrophilic chain; and
a calcium fluorescent indicator.

12. The test kit according to claim 11, further comprising, as the test substance, any one selected from an antigen, an antigenic protein, an antigenic peptide, a vaccine, and a pathogen.

13. The test kit according to claim 11, wherein the cell membrane modifier is represented by the formula (1): 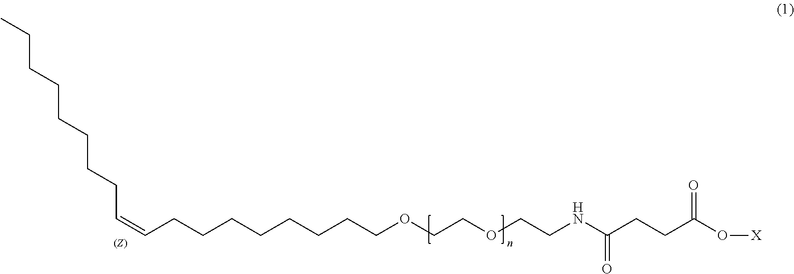
in the formula (1), X represents a reactive group, and “n” represents an integer of 20 or more and 200 or less.
